(19) United States
(12) Patent Application Publication
(10) Pub. No.: US 2012/0178645 A1
(43) Pub. Date: Jul. 12, 2012

(54) IDENTIFYING CIRCULATING TUMOR CELLS (CTCS) USING CD146 IN BREAST CANCER PATIENTS

(76) Inventors: Johannes Albert Foekens, Rotterdam (NL); Johannes Wihelmus Maria Martens, Rotterdam (NL); Jaco Kraan, Rotterdam (NL); Stefan Sleijfer, Rotterdam (NL); Bianca Mostert, Rotterdam (NL); Anita Maria Sieuwerts, Rotterdam (NL)

(21) Appl. No.: 13/380,797

(22) PCT Filed: Jun. 26, 2009
(86) PCT No.: PCT/NL2009/050378

§ 371 Date: Mar. 12, 2012

(51) Int. Cl. C40B 30/04; C12Q 1/68; G01N 33/574

(52) U.S. Cl. 506/9; 435/7.23; 435/6.11

(57) ABSTRACT

The present invention relates to a method for diagnosing cancer in a subject said method comprising the steps of providing a biological sample from a subject, and determining the expression of the MCAM gene in a circulating tumor cell (CTC) in said biological sample.

### FIELD OF THE INVENTION

The present invention relates to the field of diagnostic testing, and more particularly to diagnostics in the oncology field. The invention is useful in cancer screening, staging, monitoring for chemotherapy treatment responses, cancer recurrence or the like. More specifically, the present invention provides reagents, methods and test kits that facilitate analysis and enumeration of tumor cells, or other rare cells isolated from biological samples. The invention also provides materials and methods for assessing tumor diathesis associated molecules, such as nucleic acids, proteins and carbohydrates, thereby aiding the clinician in the design of therapeutic treatment strategies.

### BACKGROUND OF THE INVENTION

The outcome of breast cancer is largely determined by the occurrence of metastases. Currently, there is a profound lack of predictive and prognostic markers for patients with metastatic breast cancer. The detection of circulating tumor cells (CTCs) offers a new opportunity to detect metastatic disease earlier, less invasive and more reliably than currently available conventional methods do. Simultaneously, the course of disease and response to systemic therapy can be evaluated by enumerating CTCs at consecutive time points. A large variety of methods for CTC detection has been developed, but due to the rarity of these cells and the lack of a specific feature that universally distinguishes CTCs from blood cells, implementation of a suitable assay has proven to be difficult. To eliminate false-negative results, CTC detection should rely on a (set of) markers that is expressed in every cell of every tumor type, which is challenging due to the heterogeneity of marker expression between different histological subtypes and even within one tumor.

In the case of breast cancer, most assays rely on EpCAM expression on tumor cells as a marker to detect CTCs. EpCAM (epithelial cell adhesion molecule), or CD326, is a cell surface molecule that is known to be highly expressed in breast and other epithelial tumors. In a large study, the EpCAM-based CellSearch assay detected >2 CTCs in 37% of 1,316 metastatic breast cancer patient samples. In CellSearch (Veridex™, Warren, Pa.), currently the only FDA-approved assay for CTC detection, whole blood is enriched for CTCs by adding ferrofluids loaded with antibodies directed towards EpCAM. CTCs in the enriched population are stained with CK and DAPI using fluorescent antibodies, while hematopoietic cells are counterstained with CD45. The CK+/DAPI+/CD45 cells are then enumerated with an automated fluorescence microscope. Much progress has been made in establishing the enumeration of CTCs with CellSearch as a predictive and prognostic factor in breast cancer.

As mentioned, tumor heterogeneity poses a major challenge in CTC detection. In breast cancer, 5 different clinically relevant molecular subtypes have been identified by gene expression profiling. This subclassification into luminal A and B, basal, Her2-positive and normal-like tumors has prognostic and predictive value. Recently, our group reported that in contrast to the other molecular subtypes, the normal-like subtype lacks EpCAM expression and is therefore overlooked using CellSearch technology. As normal-like breast cancer cells and also EpCAM negative breast cancer cells from a molecular subtype other than normal-like are also likely to be of clinical relevance, there is a need to detect these cells as well.

### SUMMARY OF THE INVENTION

In order to solve the above problems, the present inventors hypothesized that the lower recovery rates obtained in normal-like and basal breast cancer subtypes were due to a lower membrane expression of EpCAM. This prompted the search for an alternative marker for detection of breast cancer CTC.

The present inventors have discovered that CD146 provides for an additional marker that is capable of detecting CTCs associated with breast cancer that are overlooked in the prior art procedures.

Hence, in a first aspect the present invention provides a method for diagnosing cancer in a subject said method comprising the steps of:
providing a biological sample from a subject, and
determining the expression of the MCAM gene in a circulating tumor cell (CTC) in said biological sample.

Preferably, said cancer is breast cancer.

In a preferred embodiment of a method of the invention, the method further comprises the step of comparing the level of expression of the MCAM gene to the level of expression of a suitable control gene in said CTC, such as a housekeeping gene, or to the level of expression of the MCAM gene in a suitable control cell, such as a healthy epithelial or healthy blood cell, wherein a significant increase in the level of expression of the MCAM gene relative to said control indicates that said individual has an increased risk for cancer, a metastatic cancer and/or a poor prognosis for cancer or recurrence thereof. Preferably, the MCAM gene is the gene having the sequence as described in SEQ ID NO: 1.

In another preferred embodiment, the expression of the MCAM gene is detected by detection of CD146 mRNA in said CTC, for instance by (q)RT-PCR optionally in combination with a microarray, or by detection of the CD146 protein antigen on the surface of said CTC, for instance by immunohistochemistry or FACS analysis using an anti-CD146 antibody.

In another preferred embodiment, the CTC is EpCAM-negative.

In yet another preferred embodiment, the method is for diagnosing “normal-like” breast cancer.

In a further preferred embodiment, said method comprises the step of detecting and/or isolating circulating tumor cells (CTCs). Preferably said step of detecting and/or isolating CTCs involves an assay for the detection in a circulating fluid of nucleated cells of epithelial origin other than leukocytes. In a highly preferred embodiment said assay comprises the use of a nuclear stain, preferably DAPI, a marker for epithelial cells, preferably an anti-cytokeratin antibody, and a leukocyte marker, preferably an anti-CD45 antibody.

In a highly preferred embodiment of a method of the invention said step of detecting and/or isolating CTCs further involves an assay for distinguishing CTCs from circulating endothelial cells (CECs). Preferably said assay comprises the use of a marker for endothelial cells, preferably an anti-CD34 antibody.

In another preferred embodiment, said method further comprises the step of detecting the EpCAM antigen on said CTCs.

In another preferred embodiment, said method comprises the use of a CellSearch™ assay.

In another preferred embodiment of a method of the present invention said subject is known to suffer or to have suffered from breast cancer and said method is a prognostic method for assessing the progression or risk of recurrence of the disease.

In another preferred embodiment of a method of the present invention, the method further comprised the step of determining the number of CTCs in said blood sample that express the MCAM gene and comparing said number with a statistically determined number of CTCs that do not express the MCAM gene from a group of tumor-free patient controls, and assigning a likelihood of cancer recurrence or disease progression when said number exceeds a predetermined value based on statistical averages of the number of CTCs that do not express the MCAM gene in samples from healthy subjects compared with statistical averages of CTCs that express the MCAM gene from cancer patients. Enumeration may suitably be done by using immunohistochemistry or in situ mRNA staining in combination with a FACS cell sorter.

In another aspect, the present invention provides a kit-of parts adapted for performing a method according to the present invention as described above, comprising an anti-CD146 antibody and at least one selected from:
a nuclear stain, preferably DAPI;
a marker for epithelial cells, preferably an anti-cytokeratin antibody;
a leukocyte marker, preferably an anti-CD45 antibody;
an anti-EpCAM antibody;
an marker for endothelial cells, preferably an anti-CD34 antibody;
an instruction for performing the method according to any one of claims 1-10.

In a preferred embodiment of the kit-of-parts of the invention said antibodies are labelled or stained with radioactive labels, luminescent dyes, fluorescent dyes, enzyme reagents or paramagnetic labels.

In another aspect, the present invention provides a method for determining the prognosis of cancer recurrence in a human subject suffering from breast cancer, comprising steps of
a. providing a blood sample;
b. determining a number CTCs in said blood sample according to the method of the present invention for diagnosing breast cancer as described above, and
c. comparing said number with a statistically determined number of false positive CTCs from a group of tumor-free patient controls, and assigning a likelihood of cancer recurrence when said number exceeds a predetermined value based on statistical averages of the number of false positive CTCs in samples from healthy subjects compared with statistical averages of CTCs from cancer patients.

### DESCRIPTION OF THE DRAWINGS

FIG. 1: EpCAM and CD146 membrane expression in normal-like (n), basal-like (b) and luminal (l) cell lines (A), and recovery of these cell lines with anti-EpCAM, anti-CD146 and mixed ferrofluid (B).

### DETAILED DESCRIPTION OF THE INVENTION

### Definitions

The term “identifying”, as used herein, refers to a process of establishing the identity or distinguishing character of a cell, such as exhibiting a certain surface marker or other molecular characteristic.

The term “circulating tumor cells” (CTCs) is used herein to indicate nucleated cells (DAPI+) in a circulating fluid (preferably peripheral blood) of epithelial origin (CK+) that are not leukocytes (CD45-), wherein DAPI is the nucleic acid stain 4′,6-diamidino-2-phenylindole, CK indicates the intracytoplasmic cytoskeleton protein of epithelial tissue cytokeratin, and CD45 is the antigen “protein tyrosine phosphatase, receptor type, C” (PTPRC) or leukocyte common antigen. Detection of CTCs finds important application in diagnosis and prognosis of cancer. The presence in peripheral blood of CTCs expressing on their surface the ‘Epithelial Cell Adhesion Molecule’ (EpCAM, a pan-epithelial differentiation antigen that is expressed on almost all carcinomas), (EpCAM+ CTCs) is associated with decreased progression free survival and decreased overall survival in patients treated for metastatic breast cancer. An EpCAM+ CTC count of 5 or more per 7.5 ml of blood is predictive of shorter progression free survival and overall survival.

The term “biological sample” as used herein, is used in its broadest sense as containing nucleic acids or the protein translation products thereof. A sample may comprise a bodily fluid such as blood; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue; a tissue print; a fingerprint; cells; skin, and the like. In preferred embodiments, the term refers to biological material obtained from a subject that contains cells and encompasses any material in which CTCs can be detected. A sample can be, for example, whole blood, plasma, saliva or other bodily fluid or tissue that contains cells. A preferred sample is whole blood, more preferably peripheral blood, still more preferably a peripheral blood cell fraction, still more preferably CTCs isolated or enriched from blood.

The term “antibody” as used herein refers to any of a large variety of proteins normally present in the body or produced in response to an antigen which it neutralizes, thus producing an immune response. An antibody preferably comprises immunoglobulins of the IgG subtype.

The term “nuclear stain” refers to a dye compound used to indicate the presence of a nucleus in a cell. Nuclear stains include such intercalating dyes such as acridine orange, ethidium bromide, ethidium monoazide, Hoechst dyes, propidium iodide and DAPI.

The term “fluorescent label”, as used herein, refers to a fluorophore that can be covalently attached to another molecule, such as a protein or nucleic acid, which attachment is generally accomplished by using a reactive derivative of the fluorophore that selectively binds to a functional group contained in the target molecule. Fluorescent labels include, but are not limited to fluoresceins (fluoresceins, FITC), rhodamines (FAM, R6G, TET, TAMRA, JOE, HEX, CAL Red, VIC, and ROX), Texas red, BODIPY, coumarins, cyanine dyes (thiazole orange [TO], oxazole yellow [YO], TOTO, YOYO; Cy3, Cy5), Alexa dyes, green fluorescen protein (GFP) and phycoerythrin (PE).

The term “breast cancer” refers to a malignancy that forms in tissues of the breast, usually the ducts and lobules.

The term “normal-like” breast cancer is a breast cancer representing one of the five molecular subtypes which is negative for the EpCAM marker.

The term “reacts specifically with”, as used herein, refers to the binding between an antibody and an antigen with a specificity (and generally also affinity) which is better than the binding between the same antigen and a non-specific antibody.

The term “CD146”, the abbreviation of “cluster of differentiation 146”, as used herein, refers to the CD146 protein, the 113 kDa cell adhesion molecule encoded in humans by the MCAM gene (melanoma cell adhesion molecule) (a.k.a. MUC18) located on chromosome 11 band q23.3. Two isoforms exist (MCAM long (MCAM-1), and MCAM short, or MCAM-s) which differ in the length of their cytoplasmic domain. Both isoforms are suitably used in aspects of the present invention. A representative sequence of the MCAM gene is provided as SEQ ID NO: 1 herein.

The term “gene”, as used herein refers to a DNA sequence including but not limited to a DNA sequence that can be transcribed into mRNA which can be translated into polypeptide chains. The term refers to any DNA sequence comprising several operably linked DNA fragments such as a promoter region, a 5′ untranslated region (the 5′ UTR), a coding region (which may or may not code for a protein), and an untranslated 3′ region (3′ UTR) comprising a polyadenylation site. Typically, the 5′UTR, the coding region and the 3′UTR are transcribed into an RNA of which, in the case of a protein encoding gene, the coding region is translated into a protein. The gene usually comprises introns and exons and thus a gene may include additional DNA fragments such as, for example, introns.

“Expression” refers to the transcription of a gene into structural RNA (rRNA, tRNA) or messenger RNA (mRNA) with subsequent translation into a protein.

The term “nucleic acid” as used herein, includes reference to a deoxyribonucleotide or ribonucleotide polymer, i.e. a polynucleotide, in either single-or double-stranded form, and unless otherwise limited, encompasses known analogues having the essential nature of natural nucleotides in that they hybridize to single-stranded nucleic acids in a manner similar to naturally occurring nucleotides (e. g., peptide nucleic acids). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “stringency” or “stringent hybridization conditions” refer to hybridization conditions that affect the stability of hybrids, e.g., temperature, salt concentration, pH, formamide concentration and the like. These conditions are empirically optimised to maximize specific binding and minimize non-specific binding of primer or probe to its target nucleic acid sequence. The terms as used include reference to conditions under which a probe or primer will hybridise to its target sequence, to a detectably greater degree than other sequences (e.g. at least 2-fold over background). Stringent conditions are sequence dependent and will be different in different circumstances. Longer sequences hybridise specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridises to a perfectly matched probe or primer. Typically, stringent conditions will be those in which the salt concentration is less than about 1.0 M Na⁺ ion, typically about 0.01 to 1.0 M Na⁺ ion concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1 M NaCl, 1% SDS at 37° C., and a wash in 0.1×SSC at 60° C. Hybridization procedures are well known in the art and are described in e.g. Ausubel et al, Current Protocols in Molecular Biology, John Wiley & Sons Inc., 1994.

Methods of the invention can in principle be performed by using any nucleic acid amplification method, such as the Polymerase Chain Reaction (PCR; Mullis 1987, U.S. Pat. Nos. 4,683,195, 4,683,202, en 4,800,159) or by using amplification reactions such as Ligase Chain Reaction (LCR; Barany 1991, Proc. Natl. Acad. Sci. USA 88:189-193; EP Appl. No., 320,308), Self-Sustained Sequence Replication (3SR; Guatelli et al., 1990, Proc. Natl. Acad. Sci. USA 87:1874-1878), Strand Displacement Amplification (SDA; U.S. Pat. Nos. 5,270,184, en 5,455,166), Transcriptional Amplification System (TAS; Kwoh et al., Proc. Natl. Acad. Sci. USA 86:1173-1177), Q-Beta Replicase (Lizardi et al., 1988, Bio/Technology 6:1197), Rolling Circle Amplification (RCA; U.S. Pat. No. 5,871,921), Nucleic Acid Sequence Based Amplification (NASBA), Cleavase Fragment Length Polymorphism (U.S. Pat. No. 5,719,028), Isothermal and Chimeric Primer-initiated Amplification of Nucleic Acid (ICAN), Ramification-extension Amplification Method (RAM; U.S. Pat. Nos. 5,719,028 and 5,942,391) or other suitable methods for amplification of DNA. Generally, in order to detect gene expression in the form of mRNA, the mRNA is first reverse transcribed into cDNA by reverse transcription using methods well known in the art, for instance based on the use of M-MLV reverse transcriptase from the Moloney murine leukemia virus or AMV reverse transcriptase from the avian myeloblastosis virus. Subsequently, the cDNA is then amplified by using for instance the PCR reaction.

In order to amplify DNA with a small number of mismatches to one or more of the amplification primers, an amplification reaction may be performed under conditions of reduced stringency (e.g. a PCR amplification using an annealing temperature of 38° C., or the presence of 3.5 mM MgCl₂). The person skilled in the art will be able to select conditions of suitable stringency.

The primers used for amplification of nucleic acids are selected to be “substantially” complementary (i.e. at least 65%, more preferably at least 80% perfectly complementary) to their target regions present on the different strands of each specific sequence to be amplified. It is possible to use primer sequences containing e.g. inositol residues or ambiguous bases or even primers that contain one or more mismatches when compared to the target sequence. In general, sequences that exhibit at least 65%, more preferably at least 80% homology with the target DNA oligonucleotide sequences, are considered suitable for use in a method of the present invention. Sequence mismatches are also not critical when using low stringency hybridization conditions.

The detection of the amplification products can in principle be accomplished by any suitable method known in the art. The detection fragments may be directly stained or labelled with radioactive labels, antibodies, luminescent dyes, fluorescent dyes, or enzyme reagents. Direct DNA stains include for example intercalating dyes such as acridine orange, ethidium bromide, ethidium monoazide or Hoechst dyes.

Alternatively, the DNA fragments may be detected by incorporation of labelled dNTP bases into the synthesized DNA fragments. Detection labels which may be associated with nucleotide bases include e.g. fluorescein, cyanine dye or BrdUrd. mRNA expression analysis may also be performed by expression profiling, using DNA microarrays by methods well known in the art.

The term “CellSearch assay™” as used herein refers to the FDA approved cellsearch test which works by using antibodies that are joined to microscopic iron particles, called ferrofluid. These antibody/ferrofluid combinations attach very specifically to CTCs. Powerful magnets then “pull” the CTCs out of the blood sample and they are then stained with additional bio-molecules and chemicals so that they can be positively identified as CTCs. The cellsearch(TM) test can accurately predict prognosis much earlier than the prostate specific antigen serum tumor marker test.

The term “progression of a disease” refers to a cancer that continues to grow or spread.

The term “false positive CTCs” refers to cells not being cancer cells which stain positively for CD146.

The detection of circulating tumor cells has proven its value as a prognostic marker in metastatic breast cancer, being related to both prognosis in terms of progression-free survival and overall survival. Even more important for daily clinical practice, a decline or rise in circulating tumor cells at first follow-up of therapy compared to baseline CTC level, predicts for early relapse in the neoadjuvant, adjuvant and metastatic setting. Therefore, monitoring of response to anti-tumor therapy is another potential application for CTC detection. When compared to conventional radiographic imaging at 10 weeks after start of therapy, CTC levels measured at 4 weeks were more informative for overall survival.

The implementation of CTC detection into clinical practice as a predictive and prognostic factor is dependant upon the ability of the test to detect CTCs in all patients with breast cancer. The choice of a marker to enrich for tumor cells in whole blood is of vital importance in this matter. We have previously shown that, in contrast to the other 4 molecular subtypes, normal-like breast cancer cell lines lack EpCAM expression and are missed by EpCAM-dependant CTC assays. This finding urged the need to identify an additional marker to detect EpCAM-negative breast cancer cells such as normal-like breast cancer, as with EpCAM enrichment alone, at least 5-10% of breast cancers could be missed. CD146, or MUC18, is expressed on melanoma cells and a subset of activated T-cells, among others. Taking normal-like breast cancer cell lines as a model for EpCAM-negative breast cancer cells, the present inventors have shown that CD146 is present on a large majority of normal-like breast cancer cell lines and is a suitable marker to detect normal-like breast cancer cells in blood. While CD146 is also present on endothelial cells, which can be more abundant in cancer patients than in healthy donors, the present inventors have revealed that CD34 is an excellent marker to distinguish CECs from CTCs. EpCAM co-enriches predominantly B lymphocytes, in contrast to the activated T-cells targeted by CD146, but both of these cell types can be identified according to their expression of CD45. The combined use of anti-CD146 and anti-EpCAM ferrofluids enables the detection of all molecular subtypes of breast cancer, while the specificity of the assay is not compromised with the addition of CD34.

In patients with metastasized breast cancer, the addition of CD146 to EpCAM as an enrichment marker led to additionally detected CTCs in 7 out of 10 patients. mRNA expression profiling from CD146-enriched whole blood containing CD146+ CTCs revealed, besides high expression of CEC-specific genes, high expression of multiple epithelial-specific genes showing that cancer cells were indeed enriched by anti-CD146 ferrofluids.

Thus, in methods of the present invention, gene expression determination or profiling may be used to reveal the presence of CD146+epithelial cells in these patients' blood. Such methods may include the detection in CTCs of CD146-specific mRNA, in particular it may be detected at an expression level exceeding that of a suitable control cell.

The detection and subsequent characterization of circulating tumor cells, although already well established in metastatic breast cancer, can be even more relevant when detection is possible in all patients despite tumor heterogeneity. The addition of CD146 as an enrichment marker significantly expands the panel of subtypes that can be detected and should thus be implemented into current EpCAM-based detection methods such as CellSearch (Cristofanilli et al. N Engl J Med 2004; 351: 781-791).

Hence, in a first aspect the present invention provides a method for diagnosing cancer in a subject said method comprising the steps of:
providing a biological sample from a subject, and
detecting the CD146 antigen on the surface of circulating tumor cells (CTCs) in said biological sample.

The detection of a membrane marker on a cell can be done using any suitable detection technique. Methods for the detection of membrane proteins are well known to a skilled person and include immunocytochemistry and microscopy, western blotting, preferably fluorescent microscopy and (RT-)PCR. Preferably, said detection further comprises a step of cell selection based on labelling with antibodies in combination. Such selection techniques are known to a skilled person and include techniques to enrich cell population based on specific labelling using magnetic beads and a step wherein labelled cells are separated the non labelled cells or vice versa by the provision of a strong magnetic field.

Preferably, said cancer is breast cancer.

In a preferred embodiment, said method comprises the step of detecting circulating tumor cells (CTCs). Preferably said step of detecting CTCs involves an assay for the detection in a circulating fluid of nucleated cells of epithelial origin other than leukocytes. In a highly preferred embodiment said assay comprises the use of a nuclear stain, preferably DAPI, a marker for epithelial cells, preferably an anti-cytokeratin antibody, and/or a leukocyte marker, preferably a B-cell marker, preferably an anti-CD45 or an anti-CD19 antibody.

In a highly preferred embodiment of a method of the invention said step of detecting CTCs further involves an assay for distinguishing CTCs from circulating endothelial cells (CECs). Preferably said assay comprises the use of a marker for endothelial cells, preferably an anti-CD34 antibody.

In another preferred embodiment, said method further comprises the step of detecting the EpCAM antigen on said CTCs.

In another preferred embodiment, said method comprises the use of a CellSearch™ assay.

In another preferred embodiment of a method of the present invention said subject is known to suffer or to have suffered from breast cancer and said method is a prognostic method for assessing the progression or risk of recurrence of the disease.

In another aspect, the present invention provides a kit-of parts adapted for performing a method according to the present invention as described above, comprising an anti-CD146 antibody and at least one selected from:
a nuclear stain, preferably DAPI;
a marker for epithelial cells, preferably an anti-cytokeratin antibody;
a leukocyte marker, preferably an anti-CD45 antibody;
an anti-EpCAM antibody;
a marker for endothelial cells, preferably an anti-CD34 antibody;
an instruction for performing the method according to any one of claims 1-10.

In a preferred embodiment of the kit-of-parts of the invention said antibodies are labelled or stained with radioactive labels, luminescent dyes, fluorescent dyes, enzyme reagents or paramagnetic labels.

In another aspect, the present invention provides a method for determining the prognosis of cancer recurrence in a human subject suffering from breast cancer, comprising steps of
a. providing a blood sample;
b. determining a number CTCs in said blood sample according to the method of the present invention for diagnosing breast cancer as described above, and
c. comparing said number with a statistically determined number of false positive CTCs from a group of tumor-free patient controls, and assigning a likelihood of cancer recurrence when said number exceeds a predetermined value based on statistical averages of the number of false positive CTCs in samples from healthy subjects compared with statistical averages of CTCs from cancer patients.

### EXAMPLES

### Materials & Methods

The intrinsic subtype of our well-defined panel of 41 human breast cancer cell lines (Elstrodt F, Hollestelle A, Nagel J H et al. BRCA1 mutation analysis of 41 human breast cancer cell lines reveals three new deleterious mutants. Cancer Res 2006; 66: 41-45) were determined by gene expression profiling as previously described (Sieuwerts et al. J Natl Cancer Inst 2009; 100: 61-66), which identified 10 normal-like and 5 basal cell lines (table 1).

The transcript levels of CD146 and EpCAM of cell lines were analyzed with Affymetrix GeneChip Exon 1.0 ST Arrays (Affymetrix UK Ltd., Wickham la Wooburn Grn, UK) and real-time PCR. RNA was isolated from breast cancer cell lines with the RNeasy (Micro) kit (Qiagen BV, Venlo, the Netherlands). cDNA was prepared by use of the Superscript II RNase H-kit from Invitrogen (Breda, the Netherlands). The resulting cDNA preparations were analyzed by real-time PCR with TaqMan gene expression assays and TaqMan Universal PCR Master Mix No AmpErase UNG (Applied Biosystems). PCRs were performed in a 20-μl reaction volume in a Mx3000P Real-Time PCR system (Stratagene, Amsterdam, the Netherlands). Expression of HMBS, HOPRT1, and GUSB was used as a reference to control sample loading and RNA quality, as described previously (Sieuwerts et al., Clin Cancer Res 2005; 11: 7311-7321).

Cultured human breast cancer cell lines were incubated with the following fluorochrome-conjugated monoclonal antibodies: CD34 conjugated with FITC (clone 8G12; BD Biosciences, San Jose, Calif.), CD146 conjugated with PE (clone P1H12; BD Biosciences) and EpCAM conjugated with FITC (clone EBA-1; BD Biosciences). Cells were then analyzed on a Canto flow cytometer (BD Biosciences). Unstained cells were used as a negative control. Blood samples containing EDTA (7.5-mL aliquots of blood) from a single healthy donor were obtained from CellSave Preservative Tubes (Veridex LCC). To each sample, 10 μL of a cell suspension containing 25-75 cultured cells from the indicated subtype of human breast cancer was added. To determine the actual viable cell number, a 100-μL aliquot of the cultured cells was incubated with 10 μL of 7AAD (1 μg/mL) and 100 μL of fluorescent beads (Beckman-Coulter, Inc., Miami, Fla.). After 15 minutes of incubation at room temperature, 2 mL of phosphate-buffered saline was added, and samples were analyzed on a Calibur flow cytometer (BD Biosciences). At least 10,000 beads were acquired to estimate the number of 7AAD-negative (viable) cells. The efficiency of retrieving the tumor cells was controlled by counting the exact number of viable cells that were drawn in triplicate by light microscopy after serial dilution.

To establish the number of circulating tumor cells recovered, samples were processed on the CellTrack AutoPrep analyzer (Veridex LCC) with the CellSearch circulating tumor cell enumeration kit (Veridex LCC). For the detection of cancer cells with CD146, anti-CD146 loaded ferrofluid from the CellSearch Circulating Endothelial Cell enumeration kit (Veridex LCC) was used, in a volume equivalent to the volume of anti-EpCAM loaded ferrofluids that is used. As CD146 enriches for circulating endothelial cells (CECs), and CECs have been described to express cytokeratin 18 (Cancer Genome Anatomy Project SAGE Genie), an additional marker to exclude CECs was needed. CD34 conjugated with FITC (clone 8G12; BD Biosciences) was added to the CTC enumeration kit to differentiate between CD146+ CTCs and CD146+ CECs according to the manufacturer's instructions. The number of circulating tumor cells (i.e., cells stained with the nuclear dye, 4′,6-diamidino-2-phenylindole, that are positive for cytokeratin 8,18 or 19 or pan-cytokeratin, and negative for CD45 and CD34) was determined on the CellSpotter analyzer (Veridex LCC), according to the manufacturer's instructions.

Ten patients with metastasized breast cancer signed informed consent to have 30 ml of blood drawn by vena puncture. For each patient, circulating tumor cells were enumerated in 7.5 ml of blood after EpCAM, CD146 and EpCAM and CD146 enrichment. For patients with CD146+ CTCs, an additional 30 ml of blood was drawn for gene expression studies. 7.5m1 of blood was enriched on the CellTrack™ AutoPrep Analyzer (Cell-Search CTC profile kit) with EpCAM, CD146 and mixed ferrofluid. After removal of the supernatant using a MagCellect Magnet (R&D Systems, Minneapolis, USA), the CellSearch-enriched cells were lysed by adding 250 ul of Qiagen RNeasy RLT Lysis buffer (Qiagen BV, Venlo, The Netherlands). The RNA lysate was stored at −80° C. immediately. cDNA was synthesized with the High Capacity cDNA Archive kit from Applied Biosystems (ABI),Nieuwerkerk a/d IJssel, The Netherlands. The resulting pre-amplified cDNA preparations were analyzed by real-time PCR in a 20 ul reaction volume in a Mx3000P™ Real-Time PCR System (Stratagene, Amsterdam, The Netherlands), using TaqMan™ Gene

Expression Assays in combination with TaqMan Universal PCR Master Mix No AmpErase UNG (ABI) according to the manufacturer's instructions. Levels of HMBS, HPRT1 and GUSB were used to control sample loading and RNA quality, as described previously (Sieuwerts et al. Clin Cancer Res 2005; 11: 7311-7321).

### Results

We determined the mRNA expression levels of CD146 in 41 well-described breast cancer cell lines by Affymetrix micro-array. This cell line panel consists of 10 normal-like, 5 basal-like, 5 erbb2 and 21 luminal breast cancers. Of the 10 normal-like cell lines, 7 expressed CD146 mRNA on a high level (Table 1). Using qRT-PCR, 8 cell lines expressed CD146 at a high level (Table 1).

To confirm that the CD146 mRNA expression resulted in CD146 protein expression, we evaluated CD146 membrane status by flow cytometry (Table 1). Eight of 10 normal-like cell lines had CD146 membrane expression at a level likely to be detectable using CellSearch technology.

TABLE 1
CD146 mRNA and CD146 and CD34 protein expression in
normal-like and basal-like breast cancer cell lines.

| | | CD146 | | CD146 | CD34 | EpCAM |
|---|---|---|---|---|---|---|
| | | mRNA | CD146 mRNA | membrane | membrane | membrane |
| | Intrinsic | expression | expression | expression | expression | expression |
| Cell line | subtype | Affymetrix | qRT-PCR | FACS s/n* | FACS s/n* | FACS s/n* |
| SUM102PT | Normal-like | 569 | 0.6285 | 20-200 | <5 | <5 |
| SK-BR-7 | Normal-like | 708 | 0.2736 | 20-200 | <5 | <5 |
| MDA-MB-157 | Normal-like | 420 | 0.2300 | <5 | <5 | 20-200 |
| Hs578T | Normal-like | 235 | 0.1416 | 5-20 | <5 | <5 |
| SUM159PT | Normal-like | 281 | 0.0526 | 20-200 | <5 | <5 |
| MDA-MB-231 | Normal-like | 196 | 0.0238 | 20-200 | <5 | <5 |
| SUM1315MO2 | Normal-like | 44 | 0.0315 | 5-20 | <5 | <5 |
| MDA-MB-436 | Normal-like | 65 | 0.0039 | 5-20 | <5 | <5 |
| BT549 | Normal-like | 59 | 0.0027 | 5-20 | <5 | <5 |
| SUM149PT | Basal-like | 301 | 0.0292 | 20-200 | <5 | 5-20 |
| MDA-MB-468 | Basal-like | 60 | 0.0005 | <5 | <5 | 20-200 |
| BT20 | Basal-like | 53 | 0.0008 | <5 | <5 | 20-200 |
| SUM229PE | Basal-like | 49 | 0.0063 | 20-200 | <5 | 20-200 |
| HCC1937 | Basal-like | 31 | 0.0010 | <5 | <5 | 20-200 |

*s/n: signal to noise ratio, with s/n > 5 considered positive

We again confirmed that of these 10 normal-like cell lines, only 2 are likely to be detected based on EpCAM expression (Table 1).

CD34 proved to be a suitable marker to distinguish CTCs from CECs, as none of the normal-like or basal cell lines express CD34 (Table 1).

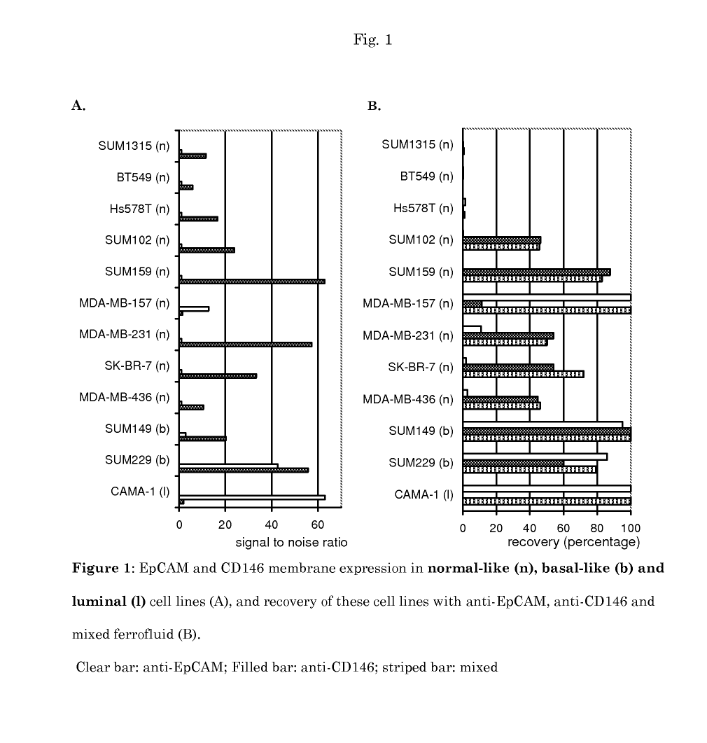

To test whether normal-like breast cancer cells could be detected in HD blood using CellSearch with anti-CD146 loaded ferrofluids, a fixed amount of cells from normal-like and basal cell lines was spiked into 7.5 ml HD blood. As a control, a fixed amount of luminal cells (CAMA-1) was spiked into 7.5 ml HD blood. Additionally, a mixture of both each basal and the luminal cell line as well as a mixture of each normal-like and the luminal cell line was spiked into HD blood. Normal-like cell lines were recovered with anti-CD146 loaded ferrofluids, but not with anti-EpCAM loaded ferrofluids (FIG. 1). A combination of anti-CD146 and anti-EpCAM ferrofluid was able to detect all CTCs from a mixture of spiked normal-like and luminal cell lines. For one of 5 basal cell lines, the addition of the anti-CD146 ferrofluid resulted in an additional recovery of 8% (FIG. 2). As expected, CAMA-1 could only be recovered with EpCAM ferrofluid, but the use of mixed ferrofluid did not result in loss of sensitivity (FIG. 3). In the samples enriched with anti-CD146 ferrofluid, either in mixture of alone, a constant number of CK+/DAPI+/CD45−/CD34+ cells was identified in each sample, accounting for a subset of CECs from the HD (data not shown).

In patients with metastasized breast cancer, the addition of CD146 to

EpCAM as an enrichment marker led to additionally detected CTCs in 7 out of 10 patients (Table 2). mRNA expression profiling from CD146-enriched whole blood containing CD146+ CTCs revealed, besides high expression of CEC-specific genes, high expression of multiple epithelial-specific genes showing that cancer cells were indeed enriched by anti-CD146 ferrofluids.

TABLE 2
CTC counts with EpCAM, CD146 and mixed ferrofluid for
10 metastatic breast cancer patients, and primary tumor
characteristics for patients with CD146+ CTCs

| | CTC count | | |
|---|---|---|---|
| | EpCAM+ | CD146+ | EpCAM/CD146+ |
| Patient 1 | 15 | 1 | 17 |
| Patient 2 | 19 | 1 | 25 |
| Patient 3 | 0 | 4 | 1 |
| Patient 4 | 38 | 5 | 20 |
| Patient 5 | 47 | 2 | 30 |
| Patient 6 | 2 | 1 | 1 |
| Patient 7 | 28 | 5 | 40 |
| Patient 8 | 0 | 0 | 0 |
| Patient 9 | 54 | 0 | 75 |
| Patient 10 | 0 | 0 | 0 |

### Discussion

The detection of circulating tumor cells has proven its value as a prognostic marker in metastatic breast cancer, being related to both progression-free survival and overall survival. Even more important for daily clinical practice, a decline or rise in circulating tumor cells at first follow-up of therapy compared to baseline CTC level, predicts for early relapse in the neoadjuvant, adjuvant and metastatic setting. The monitoring of response to anti-tumor therapy is another potential application for CTC detection. When compared to conventional radiographic imaging at 10 weeks after start of therapy, CTC levels measured at 4 weeks were more informative for overall survival.

The implementation of CTC detection into clinical practice as a predictive and prognostic factor is dependant upon the ability of the test to detect CTCs in all patients with breast cancer. The choice of a marker to enrich for tumor cells in whole blood is of vital importance in this matter. We have previously shown that, in contrast to the other 4 molecular subtypes, normal-like breast cancer cell lines lack EpCAM expression and are missed by EpCAM-dependant CTC assays. This finding urged the need to identify an additional marker to detect EpCAM-negative breast cancer cells such as these normal-like breast cancer, as with EpCAM enrichment alone, at least 5-10% of breast cancers could be overlooked. CD146, or MUC18, is expressed on melanoma cells and a subset of activated T-cells, among others. We have shown that CD146 is present on a large majority of normal-like breast cancer cell lines and is a suitable marker to detect normal-like breast cancer cells in blood. While CD 146 is also present on endothelial cells, which can be more abundant in cancer patients than in healthy donors, CD34 is an excellent marker to distinguish CECs from CTCs. EpCAM co-enriches predominantly B lymphocytes, in contrast to the activated T-cells targeted by CD146, but both of these cell types can be identified according to their expression of CD45. The combined use of anti-CD146 and anti-EpCAM ferrofluids enables the detection of all molecular subtypes of breast cancer, while the specificity of the assay is not compromised with the addition of CD34.

In patients with metastasized breast cancer, the addition of CD 146 to EpCAM as an enrichment marker led to additionally detected CTCs in 7 of 10 patients. Gene expression profiling confirmed the presence of CD 146+ epithelial cells in these patients' blood.

The detection and subsequent characterization of circulating tumor cells, although already well established in metastatic breast cancer, can be even more relevant when detection is possible in all patients despite tumor heterogeneity. The addition of CD 146 as an enrichment marker significantly expands the panel of subtypes that can be detected and should thus be implemented into current EpCAM-based detection methods such as CellSearch (Cristofanilli et al. N Engl J Med 2004; 351: 781-791).

| SEQ ID NO. 1 | |
|---|---|
| 1 | ctgcaggtaa cggatcagcg ctgccgggat cctttcaatc atcaggaaca gcaacaggtt |
| 61 | tgcagggtca ggctggggac cctcgcccat taactctttc ttctccctgt ttctttctct |
| 121 | taggtgaggg gaaactgagt tccagggtag gctccagagt gaagagggaa gaaacatgat |
| 181 | tctcaaggcc aggtctggac aagtgtgaac accttgggcc tgcgaattca gccccctcct |
| 241 | tcctttctct ggtcaaaggc tagacttgca ggagcttgcg tttgaaggga cagcccagaa |
| 301 | ggcatcgtct gcactcccca tacaggtact tctgggtctg tgggactggc gcagggttct |
| 361 | tctcccaaag ctgccagcac tgaggctgag gcagtgtcag gccggcggca gcggcagtgg |
| 421 | tgcaatcgtt ctgggaagga tagtggccgg cctgaattct ctgtggcaag ggaggggagc |
| 481 | ccaagtggga ggccccttgg ggacaccgag gaccaggtcc gctactgctc ctcccccagg |
| 541 | aggtccccta ggggctacat tggctggcag gggctgagca gcggtgagcc tggctggctt |
| 601 | cgacccgggg cgactccggg catccgggac agcttctcct cgctgccacc tcggccagtc |
| 661 | agaccccgag acacctgtca ctaccccctc agccttccca agccaggagc ctgggagtcc |
| 721 | ggctctggcc tacctccggc agcgctccta ggcgcacgtc ccgggctggc ggcgccgggg |
| 781 | cccgccccct agggctgcgg cgcgcggggc gggggctggg ggctgcgcgg ggcggggcgg |
| 841 | gcccgggcgc tccgggcccc ctcccccgcc cccctgacgt cagcccccgg cagcctcgag |
| 901 | ctgctcactt gcgtctcgcc ctccggccaa gcatggggct tcccaggctg gtctgcgcct |
| 961 | tcttgctcgc cgcctgctgc tgctgtcctc gcgtcgcggg tgagttcgct tcgctcgcag |
| 1021 | gggccgcgcc ccggctaggg gtctgcggtg gagcgtgcca gggagcagag ccagcggcgc |
| 1081 | ggcgggtcgg ggcgttgcgt ctgggaggac gagcctcctc cctgggtccc cgatccccgg |
| 1141 | gcccttgcgc gcgagcaact cttctttgca gccagtttgc agccgggatt ctagagtatc |
| 1201 | ccgggagcag cactcggaag gcggggagga ggctgcttct gggaacgaga aggggtggag |
| 1261 | ctcagccttt cggggtgctg gggggtgggt ggtccctgag gtgctcactc tgggggcccg |
| 1321 | caattgaagc cgggcaggag gcgcagctgg ggcgcatcct caaagcctga attccgcgcc |
| 1381 | cggctgttgc tggaaaaggc agcttccttc gctggagggg gtgcgccgac ccaccccttc |
| 1441 | ccccttctgc ctgggcatca cgccaggctg gaggtgagcg agagcgggag gttcggcggc |
| 1501 | tcccgcccga gctgggcgtt ggcaggggtt gcggggcggt gtgggtcgcc tcgcgcctcc |
| 1561 | ccgagtgatg ggatcatagg ggacagagat gagggatgga ggattcccat actggacgcc |
| 1621 | cgctggctta ttttggggac cacattcagg tgggaagtgc gcccgggcac ctcggagcgt |
| 1681 | ttctccggat ccgcctggta gcagggtgct ctcgggtccc gctgcccttg tatggcccgc |
| 1741 | gcagcggtgt cgcgtgtttc tcttggctcc cattccgccg tcccgctgtc cggctgggga |
| 1801 | aggggagggc taggcaatac cagctcgctg gcctcatgcc cagtgccaac catgtcctgg |
| 1861 | ggtattccag ctactgcctc ccaggctgac tttatttctg ggaaagggct aaatcgggct |
| 1921 | ccacagttgc agccggtcca gctccaccct gccctgctct tctagtctcg ggaggagtca |
| 1981 | ggggtctgag gctctgggtt ggagacccca ccttccacct gccctccttg tccgagagcc |
| 2041 | aaggtaacaa cccaggactc ccagagtccc aggcagatgg tgtcgagtga catcacctcc |
| 2101 | tcacagggct ggcagcacgc tggcaccact gacgtcactc ctgcccactg cctggccctt |
| 2161 | gccctgaccc ctgggggaga ctctgacctc tccatcctta ccagctacct agggtggggt |
| 2221 | ccgcgggtgt gtgcggagtg ttcatggcgg tgcagctgag ggagggagca tgagaccgga |
| 2281 | acttccgcca gagttagccc gctggggagt gagggcaggg attttggagg gcagaggggt |
| 2341 | agagcagtgg tgtcttcctg gcggtggtga cacaaaaggc ctgttggccc cagcctggca |
| 2401 | catcgtttgc attcccacac tctgagctca cccggagagg agggggcctg gaaggaaagg |
| 2461 | cgttcctctt gccccgagcc tagttgcccc tttctgcccc tctacagcct cagctggagc |
| 2521 | tgtcggtgct cagtctctgc tcaatctctg cttggctcca aggacctggg atctcctggt |
| 2581 | acggggagag ggctggccca ggtggggtgg cgggtcgggg tgggggtaga gcgttcagag |
| 2641 | acagggccct ctgcagaccc tctgagtggc aggaaaaaca gctcgacgag cgctgcgagg |
| 2701 | ggaggggcgg acacgacgcg gacgtgacac agcctgggcc ccgcctccct cccccaggtg |
| 2761 | tgcccggaga ggctgagcag cctgcgcctg agctggtgga ggtggaagtg ggcagcacag |
| 2821 | cccttctgaa gtgcggcctc tcccagtccc aaggcaacct cagccatgtc gactggtttt |
| 2881 | ct |

1. A method for diagnosing breast cancer in a subject said method comprising the steps of:
providing a biological sample from a subject, and
determining the expression of the MCAM gene in a circulating tumor cell (CTC) in said biological sample,
wherein said CTC is EpCAM-negative, and
wherein said breast cancer is “normal-like” breast cancer.

2. (canceled)

3. The method of claim 1, wherein said method further comprises the step of comparing the level of expression of the MCAM gene to the level of expression of a control housekeeping gene in said CTC, or to the level of expression of the MCAM gene in a suitable control cell,
wherein a significant increase in the level of expression of the MCAM gene relative to said control indicates that said individual has an increased risk for cancer, a metastatic cancer and/or a poor prognosis for cancer or recurrence thereof.

4. The method of claim 1, wherein said MCAM gene has the sequence SEQ ID No. 1.

5. The method of claim 1, wherein said expression of the MCAM gene is detected by detection of CD146 mRNA in said CTC or by detection of the CD 146 protein antigen on the surface of said CTC.

6. (canceled)

7. The method of claim 1, wherein said method comprises the step of detecting and/or isolating circulating tumor cells (CTCs).

8. The method of claim 7, wherein said step of detecting and/or isolating CTCs employs an assay for detection in a circulating fluid of nucleated cells of epithelial origin other than leukocytes.

9. The method of claim 8, wherein said assay comprises the use of a nuclear stain, a marker for epithelial cells, and a leukocyte marker.

10. The method of claim 7, wherein said step of detecting and/or isolating CTCs further involves an assay for distinguishing CTCs from circulating endothelial cells (CECs).

11. The method of claim 10, wherein said assay comprises the use of a marker for endothelial cells.

12. The method of claim 1, wherein said method further comprises the step of detecting the EpCAM antigen on said CTCs.

13. The method of claim 1, wherein said method includes the use of a CellSearch™ assay.

14. The method of claim 1, wherein said subject is known to suffer from breast cancer and wherein said method is a prognostic method for assessing the progression of the disease.

15. The method of claim 1 further comprising the step of determining the number of CTCs in said blood sample that express the MCAM gene and comparing said number with a statistically determined number of CTCs that do not express the MCAM gene from a group of tumor-free patient controls, and assigning a likelihood of cancer recurrence or disease progression when said number exceeds a predetermined value based on statistical averages of the number of CTCs that do not express the MCAM gene in samples from healthy subjects compared with statistical averages of CTCs that express the MCAM gene from cancer patients.

16. A kit-of parts adapted for performing a method according to claim 1, comprising instructions performing said method and
an anti-CD146 antibody, and/or a nucleic acid sequence capable of hybridizing under stringent conditions to CD146 mRNA, and at least one additional component selected from he group consisting of:
a nuclear stain, preferably DAPI;
a marker for epithelial cells, preferably an anti cytokeratin antibody;
a leukocyte marker, preferably an anti CD45 antibody;
an anti-EpCAM antibody;
a nucleic acid sequence capable of hybridizing under stringent conditions to EpCAM mRNA; and
a marker for endothelial cells.

17. The method of claim 5 wherein said control cell is a healthy epithelial cell.

18. The method of claim 9 wherein said nuclear stain is DAPI.

19. The method of claim 9 wherein said marker for epithelial cells is an anti-cytokeratin antibody.

20. The method of claim 9 wherein said leukocyte marker is an anti-CD45 antibody.

21. The method of claim 11 wherein the marker for endothelial cells is an anti-CD34 antibody.

22. The kit of claim 16 wherein said nuclear stain is DAPI.

23. The kit of claim 16 wherein said marker for epithelial cells is an anti-cytokeratin antibody.

24. The kit of claim 16 wherein said leukocyte marker is an anti-CD45 antibody.

25. The kit of claim 16 wherein the marker for endothelial cells is an anti-CD34 antibody.
